SEQUENCE LISTING (5 sequences)

SEQ ID NO: 1 - protein, 170 aa, Escherichia coli
MSQVSTEFIPTRIAILTVSNRRGEEDDTSGHYLRDSAQEAGHHVVDKAIVKENRYAIRAQVSAWIASDDVQVVLITGGTGLTEGDQAPEALLPLFDREVEGFGEVFRMLSFEEIGTSTLQSRAVAGVANKTLIFAMPGSTKACRTAWENIIAPQLDARTRPCNFHPHLKK

SEQ ID NO: 2 - protein, 170 aa, Escherichia coli; identical to SEQ ID NO: 1

SEQ ID NO: 3 - protein, 161 aa, Escherichia coli
MSQLTHINAAGEAHMVDVSAKAETVREARAEAFVTMRSETLAMIIDGRHHKGDVFATARIAGIQAAKRTWDLIPLCHPLMLSKVEVNLQAEPEHNRVRIETLCRLTGKTGVEMEALTAASVAALTIYDMCKAVQKDMVIGPVRLLAKSGGKSGDFKVEADD

SEQ ID NO: 4 - protein, 81 aa, Escherichia coli
MIKVLFFAQVRELVGTDATEVAADFPTVEALRQHMAAQSDRWALALEDGKLLAAVNKTLVSFDHPLTDGDEVAFFPPVTGG

SEQ ID NO: 5 - protein, 150 aa, Escherichia coli
MAETKIVVGPQPFSVGEEYPWLAERDEDGAVVTFTGKVRNHNLGDSVKALTLEHYPGMTEKALAEIVDEARNRWPLGRVTVIHRIGELWPGDEIVFVGVTSAHRSSAFEAGQFIMDYLKTRAPFWKREATPEGDRWVEARESDQQAAKRW

(19) United States
(12) Patent Application Publication
(10) Pub. No.: US 2022/0184147 A1
(43) Pub. Date: Jun. 16, 2022

(54) BACTERIAL ENZYMATIC CONVERSION OF ANTHRACYCLINE CHEMOTHERAPEUTICS TO REDUCE TOXICITY AND PROMOTE DIVERSITY AMONG THE INTESTINAL MICROBIOTA

(71) Applicants: Northwestem University, Evanston, IL (US); Ann and Robert H. Lurie Children's Hospital of Chicago, Chicago, IL (US)

(72) Inventors: Ryan A. Blaustein, Evanston, IL (US); Patrick C. Seed, Chapel Hill, NC (US); Erica M. Hartmann, Chicago, IL (US)

(21) Appl. No.: 17/436,501

(22) PCT Filed: Mar. 4, 2020
(86) PCT No.: PCT/US2020/021033

§ 371 Date: Sep. 3, 2021

Related U.S. Application Data
(60) Provisional application No. 62/813,363, filed on Mar. 4, 2019.

(51) Int. Cl. A61K 35/741; C12N 1/20; A61K 31/704; A61P 1/14

(52) U.S. Cl. CPC A61K 35/741; A61P 1/14; A61K 31/704; C12N 1/20

(57) ABSTRACT

Abstract: Disclosed are methods and compositions for treating a subject where the subject is undergoing or is about to undergo treatment with an anthracycline chemotherapeutic. In the disclosed methods, a subject may be administered an anthracycline chemotherapeutic and the subject further may be administered a detoxifying therapeutic agent that detoxifies the anthracycline chemotherapeutic, such as one or more enzymes that catalyze metabolism of the anthracycline chemotherapeutic or one or more probiotic organisms that express the one or more enzymes that catalyze metabolism of the anthracycline chemotherapeutic.

### CROSS-REFERENCE TO RELATED PATENT APPLICATIONS

The present application claims the benefit of priority under 35 U.S.C. § 119(e) to U.S. Provisional Application No. 62/813,363, filed on Mar. 4, 2019, the content of which is incorporated herein by reference in its entirety.

### STATEMENT REGARDING FEDERALLY SPONSORED RESEARCH OR DEVELOPMENT

This invention was made with government support under grant TR001423 awarded by the National Institutes of Health. The government has certain rights in the invention.

### BACKGROUND

The field of the invention relates to methods and compositions for treating a subject where the subject is undergoing or is preparing to undergo treatment with an anthracycline chemotherapeutic and the disclosed methods and compositions reduce negative side-effects of treatment with the anthracycline chemotherapeutic. In particular, the field of the invention relates to methods and compositions for treating cancer in a subject in need thereof by administering to the subject an anthracycline chemotherapeutic, such as doxorubicin, and further by administering to the subject an additional therapeutic agent that detoxifies the anthracycline chemotherapeutic, such as one or more enzymes that catalyze metabolism of the anthracycline chemotherapeutic or one or more probiotic organisms that express the one or more enzymes that catalyze metabolism of the anthracycline chemotherapeutic.

The treatment of cancer involves a wide range of interventions including chemotherapeutics and antibiotics that cause undesirable side effects. These effects include mucositis and microbiome alterations (i.e., dysbiosis) that may precede the emergence of antibiotic-resistant organisms and invasive infections. Pediatric cancer patients receive antibiotics and chemotherapy that, consequently, put then at an increased risk for developing intestinal microbiota dysbiosis and difficult-to-treat antibiotic-resistant infections while limiting the amount of active bioavailable chemotherapeutic. Additional effects of microbiome alterations include additional health complications including asthma, diabetes, and obesity.

Natural bacterial products convert chemotherapeutics, antibiotics, and other medicinal agents into metabolites that lack toxicity and thus reduce their detrimental effects. (See Yan A, Culp E, Perry J, Lau J T, MacNeil L T, Surette M G, Wright G D. Transformation of the anticancer drug doxorubicin in the human gut microbiome. ACS Infect Dis (2018), 4(1): 68-76; the content of which is incorporated herein by reference in its entirety). Using an in vitro model system, here we demonstrate that biotransformation of doxorubicin (i.e., an anthracycline used in treating leukemia and lymphoma) impacts the microbiome and, in turn, may have clinical implications for mediating drug efficacy. We also describe the bacterial enzymatic conversion of the anthracycline chemotherapeutic doxorubicin that detoxifies the drug and permits survival of drug-sensitive members of the intestinal microbial community. This enzymatic conversion mechanism may be achieved through administration of encapsulated detoxifying enzymes or through administration of probiotic organisms. Administration is predicted to limit the gastrointestinal side effects of doxorubicin and other related anthracycline chemotherapeutics and mitigate their impact on the intestinal microbiome.

### SUMMARY

Disclosed are methods and compositions for treating a subject where the subject is undergoing or is preparing to undergo treatment with an anthracycline chemotherapeutic. The disclosed methods and compositions may be utilized to reduce negative side-effects of treatment with the anthracycline chemotherapeutic. In particular, the disclosed methods and compositions may be utilized for treating a subject having cancer and reducing the negative side-effects of an anthracycline chemotherapeutic, such as doxorubicin. In the disclosed methods, a subject may be administered an anthracycline chemotherapeutic and the subject further may be administered a detoxifying therapeutic agent that detoxifies the anthracycline chemotherapeutic, such as one or more enzymes that catalyze metabolism of the anthracycline chemotherapeutic or one or more probiotic organisms that express the one or more enzymes that catalyze metabolism of the anthracycline chemotherapeutic.

### BRIEF DESCRIPTION OF THE FIGURES

FIG. 1. Illustration of the toxicity of doxorubicin on sensitive bacterial species and enzymatic conversion of doxorubicin to a less-toxic metabolite, 7-deoxydoxorubicinolone, by resistant bacterial species. After the resistant bacterial species have converted doxorubicin to 7-deoxydoxorubicinolone, sensitive bacterial can resume growth. (Compare top half of figure versus bottom half of figure).

FIG. 2. Illustration of the development of a spectrophotometric assay to measure the concentration of active, unmodified, non-transformed doxorubicin (Dox) via absorption at λ₄₈₀ versus concentration of Dox μM.

FIG. 3. Schematic representation of 3 (three) step treatment of bacterial culture. A bacterial sample is cultured in the absence of doxorubicin (i.e., community formation, generation 1) and the population of the bacterial community is analyzed via qPCR. Subsequently, the bacterial community is subjected to treatment with doxorubicin (i.e., treatment, generation 2) and the population content of the bacterial community after treatment is analyzed. Subsequently, the bacterial community is allowed to rebound (i.e., resiliency, generation 3) and the population content again is analyzed.

FIG. 4. Growth of E. coli^{R*} in the presence of Dox and concurrent reduction in the concentration of Dox. R*: resistant via efflux and enzymatic transformation of Dox.

FIG. 5. Growth of K. pneumoniae^{R*} in in the presence of Dox and concurrent reduction in the concentration of Dox. R*: resistant via efflux and enzymatic transformation of Dox.

FIG. 6. Growth of E. faecalis^{R} in in the presence of Dox without a reduction in the concentration of Dox indicating resistance only via efflux. R: resistant via efflux.

FIG. 7. Inhibition of growth of C. innocuum^{S} in the presence of Dox and no observed reduction in the concentration of Dox indicating sensitivity. S: high sensitivity to doxorubicin.

FIG. 8. Inhibition of growth of Lactobacillus^{S} in the presence of Dox and no observed reduction in the concentration of Dox indicating sensitivity. S: high sensitivity to doxorubicin.

FIG. 9A. Concentration of Dox in 50% spent media after growth of E. coli^{R*}, K pneumoniae^{R*}, or E. faecalis^{R}.

FIG. 9B. Growth of C. innocuum^{S}, Lactobacillus^{S}, and E. faecalis^{R} in 50% media from FIG. 9A. Ec(C): E. coli 0 (Control); Ec(H): E. coli 250 (High); Ef(C): E. faecalis 0 (Control); Ef(M): E. coli 100 (Medium).

FIG. 10A. Schematic representation of method for generating Community Formation (Generation 1), Treat/Disturbance (Generation 2), and Resiliency (Generation 3).

FIG. 10B. Growth of bacteria within a community formation, prior to treatment with Dox, during treatment with Dox, and after treatment with Dox.

FIG. 11A, FIG. 11B, FIG. 11C, FIG. 11D, and FIG. 11E. Bacterial growth in mixed-microbial communities composed of members pre-determined to be highly sensitive to doxorubicin (S), resistant via efflux (R), or resistant via efflux and drug-transformation (R*): FIG. 11A: C1) began with even content of model strains of C. innocuum^{S}, Lactobacillus^{S} sp., E. faecalis^{R}, E. coli^{R*}, and K. pneumoniae^{R*}. C1 began with even content of model strains of C innocuum^{S}, Lactobacillus^{S} sp., E. faecalisR, E. coli^{R*}, and K. pneumoniae^{R*}. C2 included less E. faecalis^{R}. C3 included less E. coli^{R*} . C4 included less K. pneumoniae^{R*}, and C5 included less E. coli^{R*} and K. pneumoniae^{R*}. The bacterial communities were grown in continuous batch culture and exposed to different concentrations Dox in generation 2.

FIG. 12A and FIG. 12B. Background gentamicin in culture media “fixes” K. pneumoniae to retain its biotransformation function despite thwarting cell growth. FIG. 12A: K. pneumoniae were first grown anaerobically at 37° C. in media in the presence of doxorubicin for 24 h (i.e., so initial biotransformation took place). Cultures were centrifugated into pellets, which were washed with buffer solution and resuspended in new media containing 100 μm doxorubicin and 0, 10, or 50 μm gentamicin. Inoculum was transferred to positive controls (i.e., with doxorubicin and same gentamicin treatment) and negative controls (i.e., contained doxorubicin, but no gentamicin). All samples were incubated anaerobically at 37° C. for 24 h and bacterial growth and final doxorubicin concentration (i.e., barplot) were determined. FIG. 12B: The photo shows centrifugated pellet (top), positive control (mid), and neg control (bottom) samples. The two tubes on the far right are control growth media and doxorubicin-containing growth media (i.e., red color intensity corresponds to doxorubicin concentration in solution). Overall, while the controls collectively confirm gentamicin-stalled K. pneumoniae growth preventing drug transformation, the treated pellets appear to remain functional in a gentamicin concentration-dependent manner.

### DETAILED DESCRIPTION

The presently disclosed subject matter is described herein using several definitions, as set forth below and throughout the application.

Unless defined otherwise, all technical and scientific terms used herein have the same meaning as commonly understood by one of skill in the art to which the invention pertains. Although any methods and materials similar to or equivalent to those described herein can be used in the practice or testing of the present invention, the preferred methods and materials are described herein.

Unless otherwise specified or indicated by context, the terms “a”, “an”, and “the” mean “one or more.” For example, “a component” should be interpreted to mean “one or more components.”

As used herein, “about,” “approximately,” “substantially,” and “significantly” will be understood by persons of ordinary skill in the art and will vary to some extent on the context in which they are used. If there are uses of these terms which are not clear to persons of ordinary skill in the art given the context in which they are used, “about” and “approximately” will mean plus or minus <10% of the particular term and “substantially” and “significantly” will mean plus or minus >10% of the particular term.

As used herein, the terms “include” and “including” have the same meaning as the terms “comprise” and “comprising” in that these latter terms are “open” transitional terms that do not limit claims only to the recited elements succeeding these transitional terms. The term “consisting of,” while encompassed by the term “comprising,” should be interpreted as a “closed” transitional term that limits claims only to the recited elements succeeding this transitional term. The term “consisting essentially of,” while encompassed by the term “comprising,” should be interpreted as a “partially closed” transitional term which permits additional elements succeeding this transitional term, but only if those additional elements do not materially affect the basic and novel characteristics of the claim.

As used herein, a “subject in need thereof” refers to a subject that is need of and/or my benefit by treatment with a detoxifying therapeutic agent for an anthracycline chemotherapeutic. A subject in need thereof may include a subject undergoing therapy with an anthracycline chemotherapeutic and/or preparing to undergo therapy with an anthracycline chemotherapeutic. Subjects in need thereof may include subjects having cancer which are undergoing therapy with an anthracycline chemotherapeutic and/or preparing to undergo therapy with an anthracycline chemotherapeutic. Subject in need thereof may include subjects having cancers that may include, but are not limited to, leukemias, lymphomas, breast cancer, stomach cancer, uterine cancer, ovarian cancer, bladder cancer, and lung cancer. A subject in need thereof may include a subject having or at risk for developing mucositis, for example, mucositis resulting from treatment of the subject with an anthracycline chemotherapeutic. A subject in need thereof may include a subject having or at risk for developing decreased microbiota diversity in the gut which optionally may result from treatment of the subject with an anthracycline chemotherapeutic.

The terms “subject,” “patient,” or “host” may be used interchangeably herein and may refer to human or non-human animals. Non-human animals may include, but are not limited to non-human primates, dogs, cats, horses, or other non-human animals.

The term anthracycline chemotherapeutic agent refers to a class of drugs originally extracted from the Streptomyces bacterium and may be used to treat diseases such as cancers. Anthracyclines act by intercalating with DNA and interfering with DNA metabolism and RNA production. As used herein the term “anthracycline chemotherapeutic” may include, but is not limited to a compound selected from doxorubicin, daunorubicin, epirubicin, and idarubicin.

Method and Compositions for Detoxifying Anthracene Chemotherapeutics

Disclosed are methods and compositions for treating a subject where the subject is undergoing or is preparing to undergo treatment with an anthracycline chemotherapeutic. The disclosed methods and compositions may be utilized to reduce negative side-effects of treatment with the anthracycline chemotherapeutic. In particular, the disclosed methods and compositions may be utilized for treating a subject having cancer and reducing the negative side-effects of an anthracycline chemotherapeutic, such as doxorubicin. In the disclosed methods, a subject may be administered an anthracycline chemotherapeutic and the subject further may be administered a detoxifying therapeutic agent that detoxifies the anthracycline chemotherapeutic, such as one or more enzymes that catalyze metabolism of the anthracycline chemotherapeutic or one or more probiotic organisms that express the one or more enzymes that catalyze metabolism of the anthracycline chemotherapeutic.

In some embodiments, the disclosed methods include treating a subject undergoing treatment with an anthracycline chemotherapeutic and/or treating a subject preparing to undergo treatment with an anthracycline chemotherapeutic. The disclosed methods typically include administering to the subject a detoxifying therapeutic agent that detoxifies the anthracycline chemotherapeutic in the gut of the subject after the anthracycline chemotherapeutic is administered to the subject.

In some embodiments of the disclosed methods, the subject is undergoing treatment with an anthracycline chemotherapeutic and/or is preparing to undergo treatment with an anthracycline chemotherapeutic, where the anthracycline chemotherapeutic is selected from the group consisting of doxorubicin, daunorubicin, epirubicin, and idarubicin. Particularly, the disclosed methods may include treating a subject that is undergoing treatment with doxorubicin (Dox) and/or the subject is preparing to undergo treatment with Dox.

In some embodiments of the disclosed methods, the subject has cancer and is undergoing treatment with an anthracycline chemotherapeutic and/or is preparing to undergo treatment with an anthracycline chemotherapeutic. The disclosed methods may include a step of administering the anthracycline chemotherapeutic to the subject. In the disclosed methods, the detoxifying therapeutic agent may be administered to the subject before, concurrently with, or after the anthracycline chemotherapeutic is administered to the subject. In some embodiments, the detoxifying therapeutic agent may be administered to the subject before, concurrently with, and after the anthracycline chemotherapeutic is administered to the subject (i.e., a course of treatment with the detoxifying therapeutic agent that spans administration of the anthracycline chemotherapeutic).

In the disclosed methods, the subject typically is administered a detoxifying therapeutic agent that detoxifies the anthracycline chemotherapeutic in the gut of the subject. In some embodiments, the detoxifying agent is and enzyme that catalyzes metabolism of the anthracycline chemotherapeutic into a less-toxic metabolite or non-toxic metabolite (e.g., 7-deoxydoxorubicinolone). Suitable enzymes may include, but are not limited to, molybdopterin-dependent enzyme. (See Yan A, Culp E, Perry J, Lau J T, MacNeil L T, Surette M G, Wright G D. Transformation of the anticancer drug doxorubicin in the human gut microbiome. ACS Infect Dis (2018), 4(1): 68-76; the content of which is incorporated herein by reference in its entirety). In some embodiments, suitable enzymes are selected from enzymes encoded by the moa operon. Enzymes encoded by the moa operon may include, but are not limited to MoaA, MoaB, MoaC, MoaD, and MoaE. The amino acid sequences of E. coli MoaA, E. coli MoaB, E. coli MoaC, E. coli MoaD, and E. coli MoaE are provided as follows:

| E. coli MoaA | |
|---|---|
| (SEQ ID NO: 1) | |
| 1 | masqltdafa rkfyylrlsi tdvcnfrcty clpdgykpsg vtnkgfltvd eirrvtrafa |
| 61 | rlgtekvrlt ggepslrrdf tdiiaavren dairqiavtt ngyrlerdva swrdagltgi |
| 121 | nvsvdsldar qfhaitgqdk fnqvmagida afeagfekvk vntvlmrdvn hhqldtflnw |
| 181 | ighrpiqlrf ielmetgegs elfrkhhisg qvlrdellrr gwihqlrqrs dgpaqvfchp |
| 241 | dyageiglim pyekdfcatc nrlrvssigk lhlclfgegg vnlrdlledd tqqqaleari |
| 301 | saalrekkqt hflhqnntgi tqnlsyigg |
| E. coli MoaB | |
| (SEQ ID NO: 2) | |
| 1 | msqvstefip triailtvsn rrgeeddtsg hylrdsagea ghhvvdkaiv kenryairaq |
| 61 | vsawiasddv qvvlitggtg ltegdqapea llplfdreve gfgevfrmls feeigtstlq |
| 121 | sravagvank tlifampgst kacrtaweni iapqldartr pcnfhphlkk |
| E. coli MoaC | |
| (SEQ ID NO: 3) | |
| 1 | msqlthinaa geahmvdvsa kaetvreara eafvtmrset lamiidgrhh kgdvfatari |
| 61 | agiqaakrtw dliplchplm lskvevnlqa epehnrvrie tlcrltgktg vemealtaas |
| 121 | vaaltiydmc kavqkdmvig pvrllaksgg ksgdfkvead d |
| E. coli MoaD | |
| (SEQ ID NO: 4) | |
| 1 | mikvlffaqv relvgtdate vaadfptvea lrqhmaaqsd rwalaledgk llaavnktlv |
| 61 | sfdhpltdgd evaffppvtg g |
| E. coli MoaE | |
| (SEQ ID NO: 5) | |
| 1 | maetkivvgp qpfsvgeeyp wlaerdedga vvtftgkvrn hnlgdsvkal tlehypgmte |
| 61 | kalaeivdea rnrwplgrvt vihrigelwp gdeivfvgvt sahrssafea gqfimdylkt |
| 121 | rapfwkreat pegdrwvear esdqqaakrw |

Suitable enzymes for the disclosed methods may include, but are not limited to E. coliMoaA, E. coli MoaB, E. coli MoaC, E. coli MoaD, and E. coli MoaE or homologs thereof present in other organisms, such as homologs of E. coli MoaA, E. coli MoaB, E. coli MoaC, E. coli MoaD, and E. coli MoaE in Klebsiella pneumoniae.

In some embodiments, the detoxifying therapeutic agent of the disclosed methods and compositions comprises one or more probiotic organisms that express one or more enzymes that catalyze metabolism of the anthracycline chemotherapeutic. In some embodiments, the one or more probiotic organisms express one or more molybdopterin-dependent enzymes.

In the disclosed methods, the subject may be administered the detoxifying therapeutic agent in any suitable manner which results in delivering a suitable amount of the detoxifying agent to detoxify the anthracycline chemotherapeutic in the gut of the subject. In some embodiments, the subject is administered a form of the detoxifying therapeutic agent which is formulated for oral administration (e.g., capsules containing the therapeutic agent which deliver the therapeutic agent to the gut of the subject). In other embodiments, the detoxifying therapeutic agent may be administered gastrointestinally (e.g., via colonoscopy or enema).

In some embodiments, the disclosed methods are performed on a subject in need thereof in order to reduce negative side-effects of treatment with an anthracycline chemotherapeutic such as doxorubicin. In some embodiments, the disclosed methods treat and/or prevent mucositis. In further embodiments, the disclosed methods promotes microbiota diversity in the subject.

Also disclosed herein are methods for preparing detoxifying therapeutic agents and detoxifying therapeutic agents prepared by the disclosed methods of preparation. In some embodiments of the disclosed methods of preparation, a detoxifying therapeutic agent is prepared by a method comprising: (a) culturing a bacterial sample obtained from a gastrointestinal tract of a subject in the presence of an anthracycline chemotherapeutic to prepare a cultured sample comprising one or more bacteria that are resistant to the anthracycline chemotherapeutic; and (b) formulating the cultured sample for administration to a subject in need thereof as the detoxifying therapeutic composition. In the disclosed methods of preparation, the anthracycline chemotherapeutic may include doxorubicin and the bacterial sample is cultured in the presence of the anthracycline therapeutic at a concentration of at least about 10 82 M, 20 μM, 30 μM, 40 μM, 50 μM, 75 μM, 100 μM, 150 ρM, 200 μM, 250 μM, or higher.

Also contemplated herein are compositions and kits comprising detoxifying therapeutic agents optionally containing and/or packaging together with an anthracycline chemotherapeutic. In some embodiments the disclosed composition and/or kits comprise: (i) a detoxifying therapeutic agent that detoxifies the anthracycline chemotherapeutic in the gut of the subject; and optionally (ii) an anthracycline chemotherapeutic.

### ILLUSTRATIVE EMBODIMENTS

The following embodiments are illustrative and should not be interpreted to limit the scope of the claimed subject matter.

### Embodiment 1

A method for treating a subject undergoing treatment with an anthracycline chemotherapeutic or a subject preparing to undergo treatment with an anthracycline chemotherapeutic, the method comprising administering to the subject a detoxifying therapeutic agent that detoxifies the anthracycline chemotherapeutic in the gut of the subject.

### Embodiment 2

The method of embodiment 1, wherein the anthracycline chemotherapeutic is selected from the group consisting of doxorubicin, daunorubicin, epirubicin, and idarubicin.

### Embodiment 3

The method of embodiment 1, wherein the anthracycline chemotherapeutic is doxorubicin.

### Embodiment 4

The method of embodiment 1, wherein the subject is undergoing treatment for cancer or is preparing to undergo treatment for cancer by administration of the anthracycline chemotherapeutic, optionally wherein the cancer is selected from leukemias, lymphomas, breast cancer, stomach cancer, uterine cancer, ovarian cancer, bladder cancer, and lung cancer.

### Embodiment 5

The method of any of the foregoing embodiments, further comprising administering the anthracycline chemotherapeutic to the subject, optionally wherein the anthracycline chemotherapeutic is administered at a dose that delivers a concentration of the anthracycline chemotherapeutic to the gut of the subject of at least about 50 μM, 75 μM, 100 μM, 150 μM, 200 μM, or 250 μM, or within a concentration range bounded by any of these values (e.g., 50-250 μM).

### Embodiment 6

The method of embodiment 5, wherein the detoxifying therapeutic agent is administered to the subject before the anthracycline chemotherapeutic is administered to the subject, optionally wherein the detoxifying therapeutic agent is delivered at least 1 day, 2 days, 3 days, 1 week, 2 weeks, 3 weeks, or 1 month prior to the anthracycline chemotherapeutic being administered to the subject.

### Embodiment 7

The method of embodiment 5, wherein the detoxifying therapeutic agent is administered to the subject concurrently as the anthracycline chemotherapeutic is administered to the subject.

### Embodiment 8

The method of embodiment 5, wherein the detoxifying therapeutic agent is administered to the subject after the anthracycline chemotherapeutic is administered to the subject, optionally wherein the detoxifying agent is administered 1 hour, 2 hours, 4 hours, 12 hours, 1 day, 2 days, 3 days, 1 week, 2 weeks, 3 weeks, or 1 month after the anthracycline chemotherapeutic is administered to the subject.

### Embodiment 9

The method of embodiment 5, wherein the detoxifying therapeutic agent is administered to the subject before, concurrently with, and after the anthracycline chemotherapeutic is administered to the subject.

### Embodiment 10

The method of any of the foregoing embodiments, wherein the detoxifying therapeutic agent comprises one or more enzymes that catalyze metabolism of the anthracycline chemotherapeutic.

### Embodiment 11

The method of embodiment 10, wherein the enzyme is a molybdopterin-dependent enzyme, optionally wherein the enzyme is encoded by a moa operon and the enzyme is MoaA, MoaB, MoaC, MoaD, MoaE, or a combination thereof.

### Embodiment 12

The method of any of the foregoing embodiments, wherein the detoxifying therapeutic agent comprises one or more probiotic organisms that express one or more enzymes that catalyze metabolism of the anthracycline chemotherapeutic, optionally wherein the probiotic organisms are bacteria and optionally are selected from Escherichia coli and Klebsiella pneumoniae.

### Embodiment 13

The method of embodiment 12, wherein the one or more probiotic organisms express one or more molybdopterin-dependent enzymes.

### Embodiment 14

The method of any of the foregoing embodiments, wherein the detoxifying therapeutic agent is administered orally and optionally formulated for delivering the detoxifying therapeutic agent to the gut of the subject.

### Embodiment 15

The method of any of the foregoing embodiments, wherein the detoxifying therapeutic agent is administered gastrointestinally, optionally via colonoscopy or enema.

### Embodiment 16

The method of any of the foregoing embodiments, wherein the method treats and/or prevents mucositis and/or promotes microbiota diversity in the subject after the subject is administered the anthracycline chemotherapeutic.

### Embodiment 17

A method for preparing a detoxifying therapeutic composition, the method comprising: (a) culturing a bacterial sample obtained from a gastrointestinal tract of a subject (i.e., the gut of the subject) in the presence of an anthracycline chemotherapeutic agent to prepare a cultured sample comprising one or more bacteria that are resistant to the anthracycline chemotherapeutic agent; and (b) formulating the cultured sample for administration to a subject in need thereof as a therapeutic composition (e.g., formulating the cultured sample as a probiotic composition for oral administration and/or gastrointestinal administration).

### Embodiment 18

The method of embodiment 17, wherein the bacterial sample is cultured in the presence of the anthracycline chemotherapeutic agent at a concentration of at least about 50 μM, 75 μM, 100 μM, 150 μM, 200 μM, or 250 μM, or within a concentration range bounded by any of these values (e.g., 50-250 μM).

### Embodiment 19

The method of embodiment 17 or 18, wherein the anthracycline chemotherapeutic agent is doxorubicin.

### Embodiment 20

The method of any of embodiments 17-19 further comprising administering the detoxifying therapeutic composition to a subject in need thereof.

### EXAMPLES

The following examples are illustrative and should not be interpreted to limit the scope of the claimed subject matter.

### Example 1—Bacterial Biotransformation of Chemotherapeutics May Promote Diversity Among the Intestinal Microbiota

Abstract Overview

Pediatric cancer patients receive antibiotics and chemotherapy that, consequently, put them at an increased risk for developing intestinal microbiota dysbiosis and difficult-to-treat antibiotic-resistant infections while limiting the amount of active bioavailable chemotherapeutic. Using an in vitro model system, we demonstrate that biotransformation of doxorubicin (i.e., an anthracycline used in treating leukemia and lymphoma) impacts the microbiome and, in turn, may have clinical implications for mediating drug efficacy and side effects.

Objectives/Goals

This study aims to test the hypothesis that bacterial biotransformation of chemotherapeutics promotes gut microbial diversity by enhancing persistence of drug-sensitive taxa.

Methods/Study Population

The impacts of doxorubicin on a model community of gut bacteria was investigated in vitro in anaerobic batch culture. The synthetic community was composed of specific members predicted by genomic analysis to be sensitive to the therapeutic (i.e., Clostridium innocuum, Lactobacillus sp.), resistant via putative biotransformation (i.e., Escherichia coli, Klebsiella pneumoniae), or resistant via putative efflux (i.e., Enterococcus faecalis). Bacterial growth was monitored in monocultures by measuring OD600 and standard plate counts, and in mixed cultures by strain-targeted qPCR. Doxorubicin concentration was detected via absorbance assay.

Results/Anticipated Results

Strains with predicted resistance to doxorubicin by drug biotransformation significantly lowered concentrations of the drug in culture media. In contrast, E. faecalis proved resistant without evidence of drug transformation. Predicted sensitive strains were growth-repressed by the doxorubicin, but able to grow in spent media where biotransformation had occurred. However, they remained growth-repressed in spent media from E. faecalis where drug transformation had not been observed. Bacterial growth kinetics in mixed batch culture were dependent on starting bacterial concentrations and timing of drug exposure.

Discussion/Significance of Impact

This work will be extended to model microbial community responses to doxorubicin as a factor of microbial interactions and extent of drug transformation prior its exposure to sensitive strains. The resulting model will have translational implications for mitigating health risks during pediatric cancer treatment.

Applications

Applications of the disclosed technology include, but are not limited to, (i) prevention and treatment of anthracycline chemotherapeutic induced mucositis; and (ii) prevention and treatment of anthracycline chemotherapeutic damage to the intestinal microbiome.

Advantages

The contemporary approach to the side effects of anthracycline chemotherapeutics is largely symptomatic treatment of the side effects. Prophylactic and preemptive approaches to mitigate the intestinal effects of anthracycline chemotherapeutics are limited. The technology in this application may be incorporated into oral formulations as purified products or administered in probiotic organisms that naturally produce or are engineered to produce the necessary detoxifying enzymes. Promoting microbiome health is distinct from previous strategies to reduce generalized anthracycline toxicity that do not specifically treat mucositis and prevent microbiome changes. These therapies include iron chelation (Dexrazoxane), vitamin A derivatives, and TLR5 agonists or immune ligands. Using bio-based strategies to promote detoxification of doxorubicin also presents the potential for improving intestinal barrier function with less modification of the immune system.

Description of Disclosed Technology

The proposed technology includes the provision of bacterial enzymes that detoxify anthracycline chemotherapeutics in the intestinal tract that have been administered orally or excreted into the intestinal tract after intravenous administration. The required detoxifying enzymes may be administered as purified enzymes in oral encapsulated formulas or in inactivated or live probiotic organisms (naturally occurring or engineered).

A spectrophotometric assay to measure the concentration of active, unmodified, non-transformed doxorubicin was adopted. Naturally-occurring human-associated bacterial strains were identified that are resistant or sensitive to the antibiotic effects of doxorubicin. Using the biotransformation assay, resistant strains of Escherichia coli and Klebsiella pneumoniae were shown to transform doxorubicin in culture. Resistant Enterococcus faecalis did not transform doxorubicin. The resistance of this strain is predicted to be due to a different mechanism not related to conversion and detoxification, likely blocking of drug entry into the cells or active efflux of the drug from the cells. Clostridium innocuum and a representative Lactobacillus species were highly sensitive to doxorubicin inhibition.

Doxorubicin-sensitive strains (C. innocuum and Lactobacillus) remain growth inhibited when grown in the spent medium of E. faecalis, which does not convert doxorubicin. In contrast, C. innocuum and Lactobacillus grow in spent media from E. coli and K. pneumoniaegrown with doxorubicin, indicating that the doxorubicin was converted and detoxified. K pneumoniae is more efficient at this process. In mixed-batch culture, drug-sensitive C. innocuum grows only following doxorubicin transformation by E. coli or K. pneumoniae, and the C. innocuum population is resilient over time in continuous culture. Overall, the doxorubicin transformation mechanism can be harnessed to remediate the antimicrobial effects of the compound, which promotes microbiota diversity and may improve intestinal health.

### Example 2—Bacterial Biotransformation of Chemotherapeutics May Promote Diversity Among the Intestinal Microbiota

Overview

Cancer treatment involves interventions including chemotherapeutics and antibiotics with undesirable side effects. Side effects may include mucositis and microbiome alterations and loss of diversity preceding the emergence of antibiotic-resistant organisms and invasive infections, among other health complications. (See Alexander J L, Wilson I D, Teare J, Marchesi J R, Nicholson J K, Kinross J M. “Gut microbiota modulation of chemotherapy efficacy and toxicity.” Nat Rev Gastroenterol Hepatol (2017), 14(6): 356-365; the content of which is incorporated herein by reference in its entirety). In addition, the loss of microbial diversity may increase risks of childhood obesity, diabetes, asthma, allergies, infection (e.g., by Clostridium difficile), and enrichment of resistance genes in the microbome.

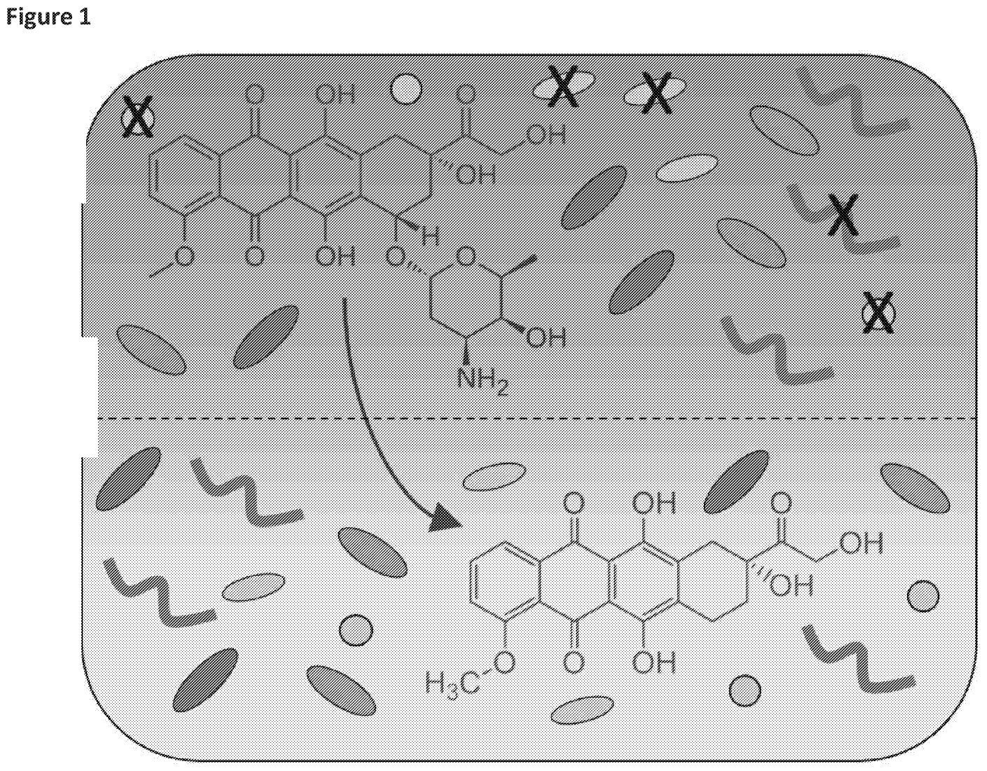

Natural products can convert medicinal agents into less-toxic metabolites. (See Yan A, Culp E, Perry J, Lau J T, MacNeil L T, Surette M G, Wright G D. Transformation of the anticancer drug doxorubicin in the human gut microbiome. ACS Infect Dis (2018), 4(1): 68-76; the content of which is incorporated herein by reference in its entirety). FIG. 1 and Scheme 1 illustrate enzymatic conversion of doxorubicin to a less-toxic metabolite (e.g., 7-deoxydoxorubicinolone).

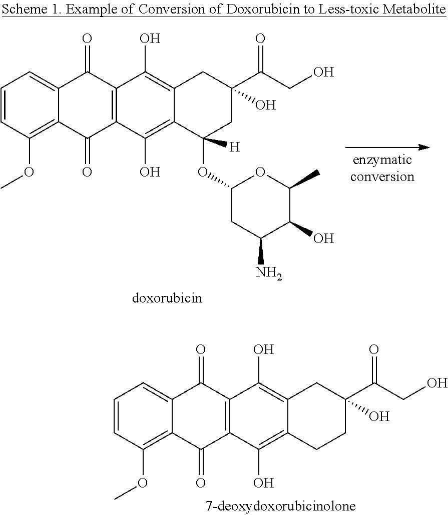 [SMILES: COc1cccc2c1C(=O)c1c(O)c3c(c(O)c1C2=O)C[C@@](O)(C(=O)CO)C[C@@H]3O[C@H]1C[C@H](N)[C@H](O)[C@H](C)O1 ; COc1cccc2c1C(=O)c1c(O)c3c(c(O)c1C2=O)C[C@@](O)(C(=O)CO)CC3]

FIG. 1 illustrates the hypothesis that bacterial transformation of doxorubicin (an anthracycline chemotherapeutic) detoxifies the drug and permits survival of drug-sensitive members of the intestinal microbial community. (See FIG. 1 top half of figure versus bottom half of figure).

Methods

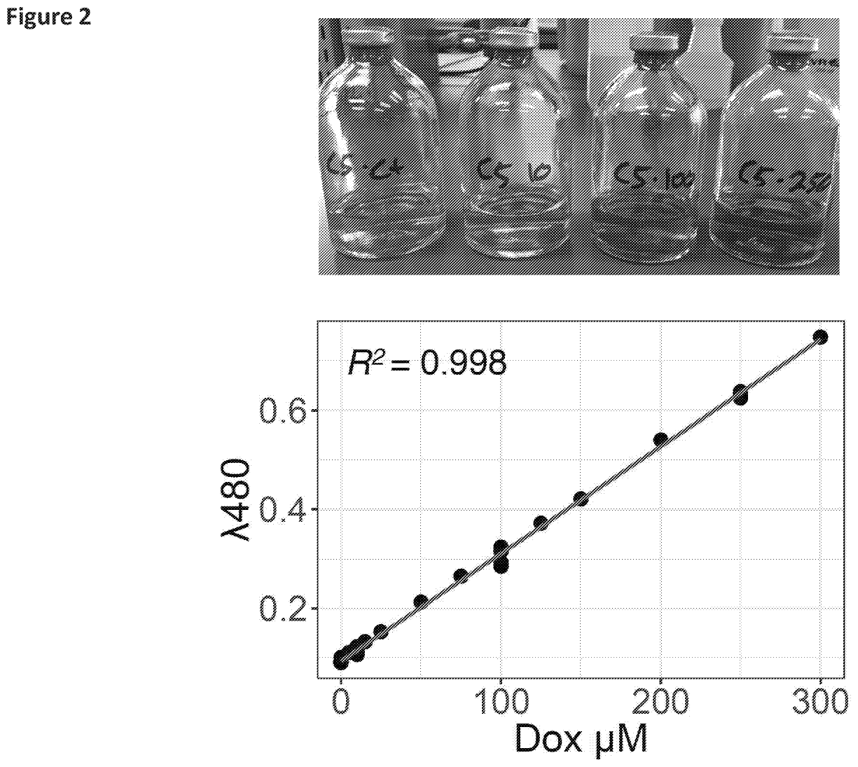

A spectrophotometric assay to measure the concentration of doxorubicin was adopted. (See FIG. 2). Our spectrophotometric assays measure the concentration of active, unmodified, non-transformed doxorubicin (Dox) via absorption at λ₄₈₀ versus concentration of Dox μM. We tested and observed good linearity from Dox concentration from 0-300 μM.

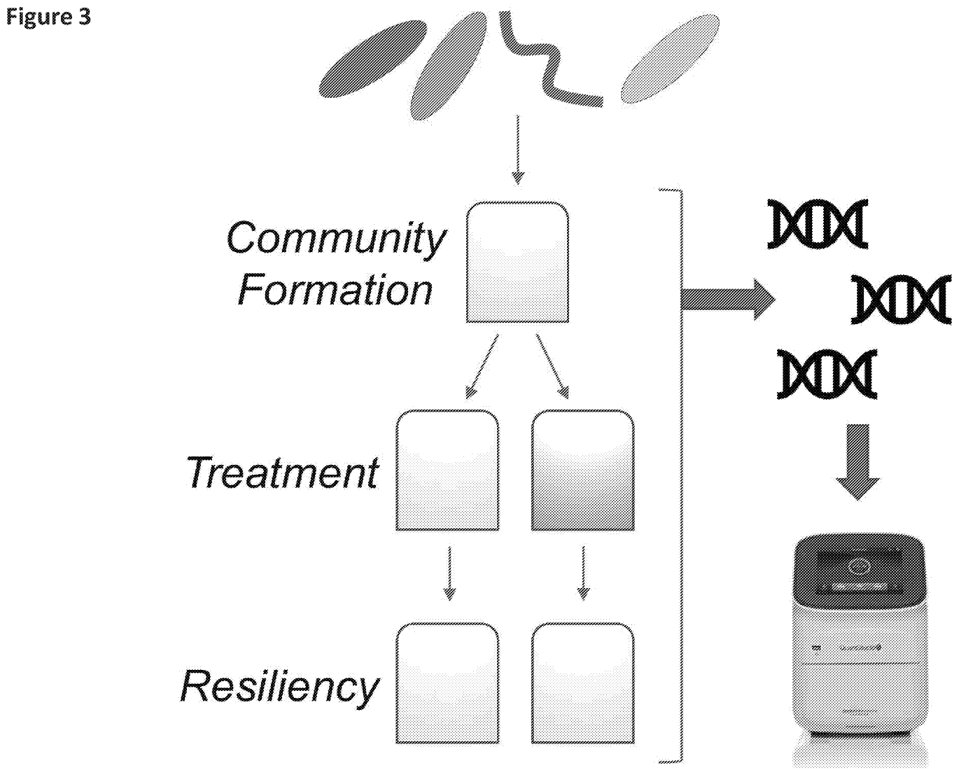

The effects of Dox on gut-associated bacteria then were investigated in vitro in monoculture and in a synthetic community. As illustrated in FIG. 3, a synthetic community was created by combining pure cultures of Escherichia coli, Klebsiella pneumonia, Enterococcus faecalis, Clostridium innocuum or Lactobacillus spp. Bacteria were identified and quantified after culture and treatment using qPCR.

Model Microbial Community Interactions with Doxorubicin

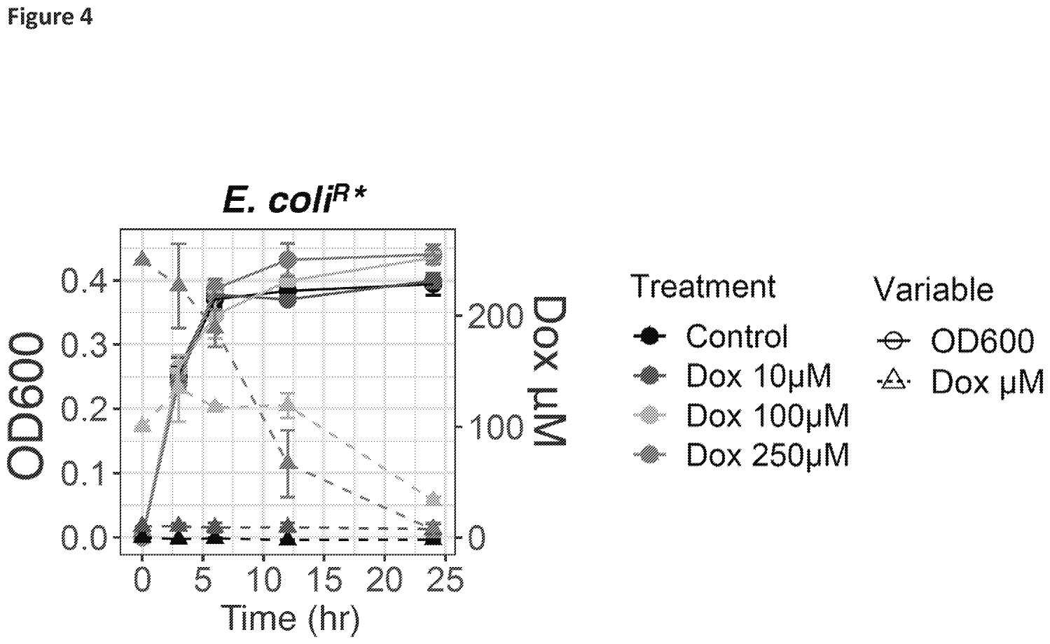

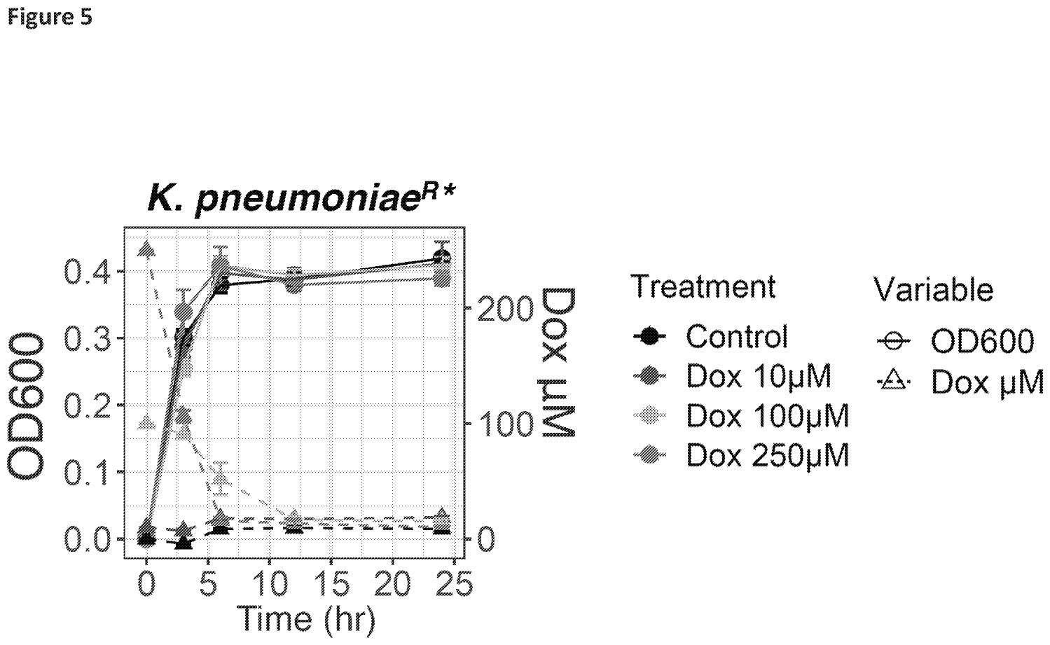

We next tested the sensitivity or resistance of Escherichia coli, Klebsiella pneumonia, Enterococcus faecalis, Clostridium innocuum or Lactobacillus spp. to Dox at various concentrations. (See FIGS. 4-8). Bacterial strains that are resistant to Dox, for example via drug efflux and/or enzymatic transformation of Dox to a less-toxic metabolite, and bacterial strains that are sensitive to antibiotic effects of Dox were identified. Based on our spectrophotometric assay, resistant strains of Escherichia coli and Klebsiella pneumoniae reduced Dox concentration in culture. (See FIGS. 4 and 5, respectively). This suggests that E. coli and K. pneumonia express enzymes which can metabolize Dox into forms that are not detected by our spectrophotometric assay. We characterized E. coli and K. pneumonia with the superscript prefix (R*) to indicate that E. coli and K. pneumonia likely are resistant due to efflux (i.e., the removal of Dox from cells and/or inability of Dox to enter cells) and enzymatic transformation of Dox.

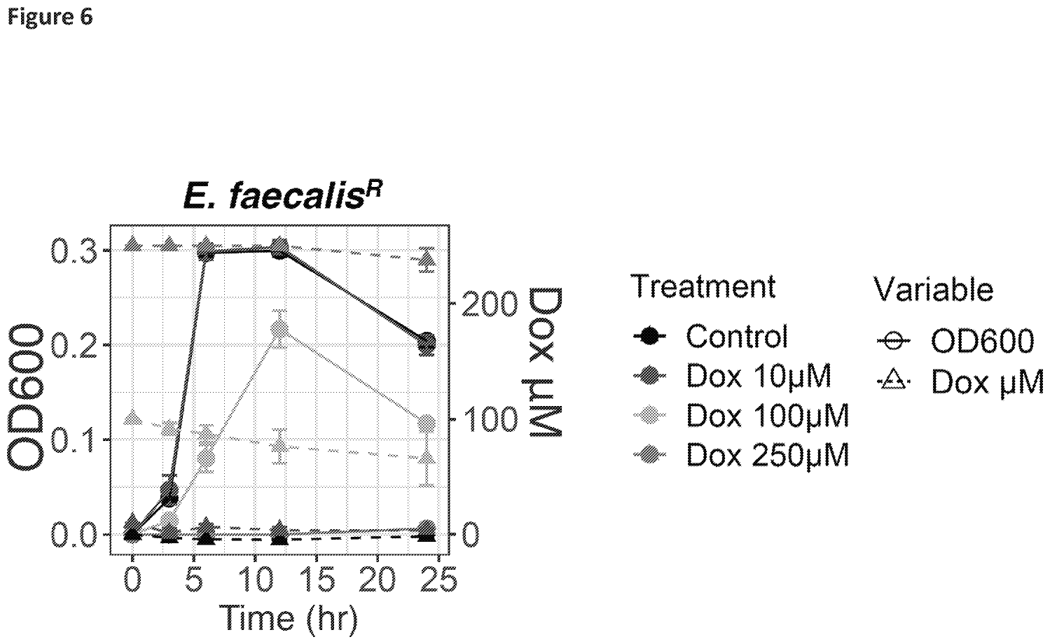

We observed that Enterococcus faecalis was resistant to Dox but did not reduce the Dox concentration in culture. (See FIG. 6). We characterized E. faecalis with the superscript prefix (R) to indicate that E. faecalis is resistant to Dox due to efflux only and not enzymatic transformation of Dox.

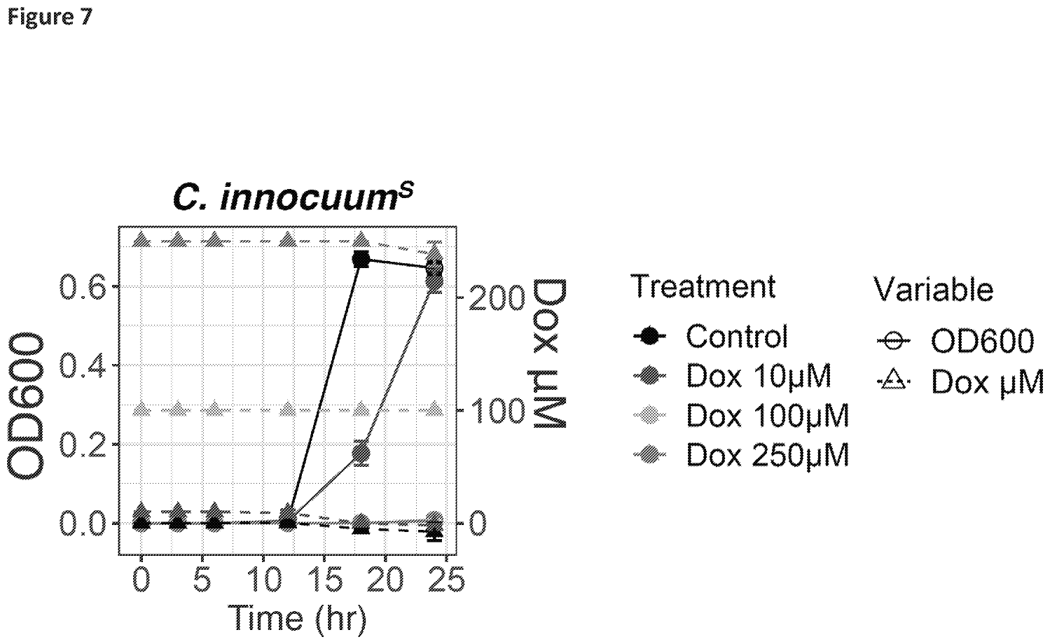

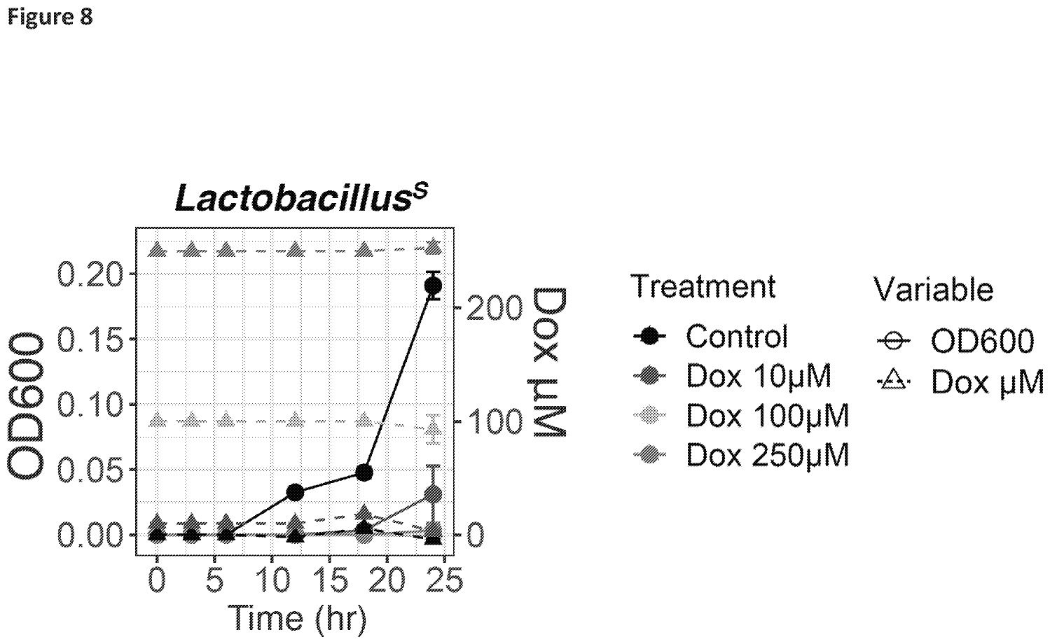

In contrast to the resistance that we observed for E. coli, K. pneumoniae, and E. faecalis, we observed that Clostridium innocuum and Lactobacillus were highly sensitive to Dox. (See FIGS. 7 and 8, respectively). We observed delayed growth of C. innocuum and Lactobacillus at a Dox concentration as low as 10 μM. and we did not observe any growth of after C. innocuum and Lactobacillus 25 hours at a Dox concentration of 100 μM.

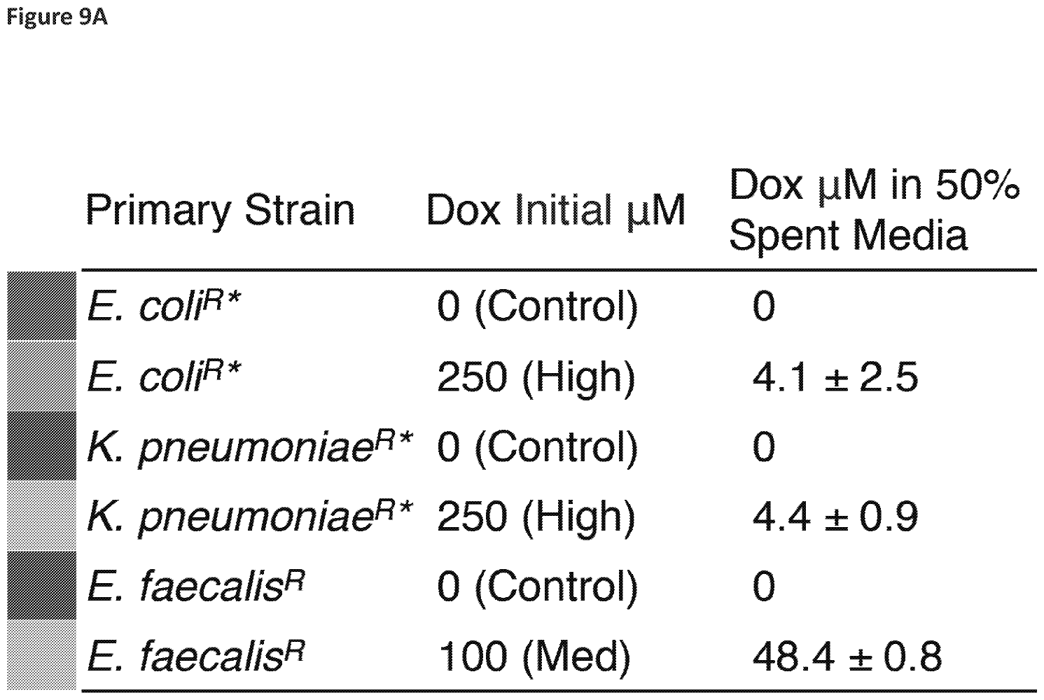

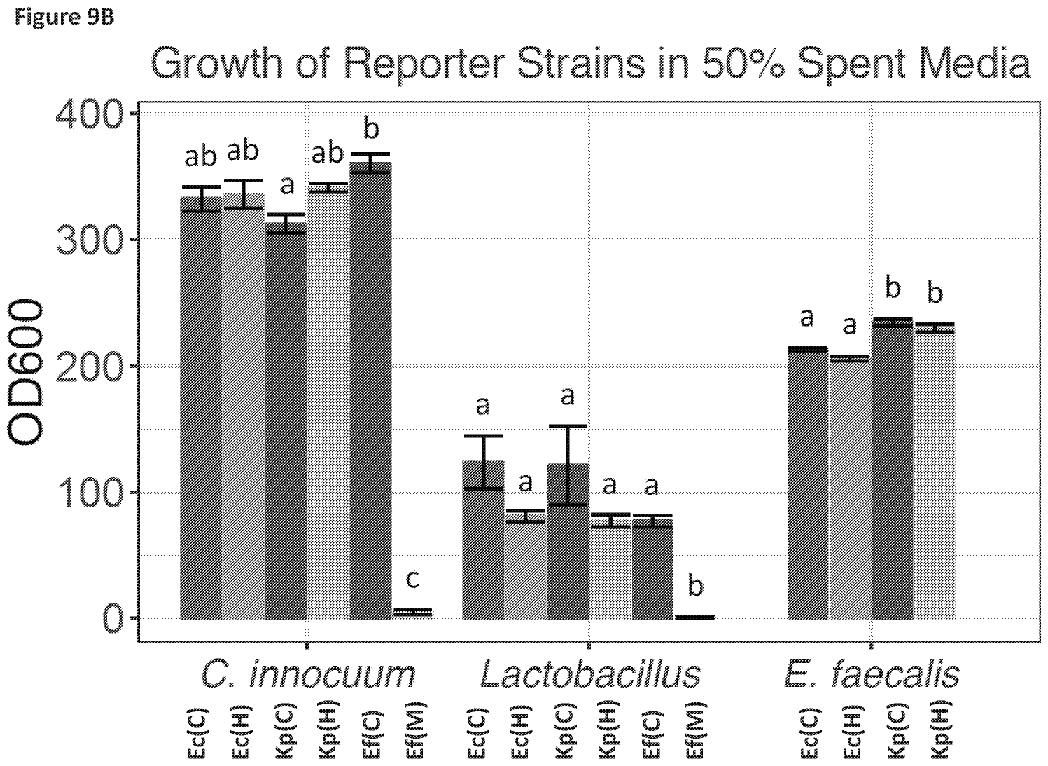

We next tested whether Dox-sensitive strains were able to grow in the spent media of resistant strains. (See FIGS. 9A and 9B). For our tests, we utilized: 50% spent media of E. coli that had been grown in the absence of Dox (Control) or in the present of 250 μM Dox (High); 50% spent media of K. pneumoniae that had been grown in the absence of Dox (Control) or in the present of 250 μM Dox (High); and 50% spent media of E. faecalis that had been grown in the absence of Dox (Control) or in the present of 100 μM Dox (Medium). As indicated in FIG. 9A, the Dox concentration in the 50% spent media of E. coli and K. pneumoniae was 4.1±2.5 μM and 4.4±0.9 respectively, which was reduced from the initial concentrations of 250 μM. The Dox concentration in the 50% spent media of E. faecalis was 48.4±0.8 which was reduced from the initial concentration of 100 μM but which was still at significant concentration level to inhibit the growth of sensitive strains.

We observed that the Dox-sensitive strains of C. innocuum and Lactobacillus were able to grow in the 50% spent media of the Dox-resistant strains E. coli and K. pneumoniae, which 50% spent media had a Dox concentration of 4.1±2.5 μM and 4.4±0.9 respectively. In contrast, we observed that the Dox-sensitive strains of C. innocuum and Lactobacillus were unable to grow in the 50% spent media of E. faecalis, which 50% spent media had a Dox concentration of 48.4±0.8 μM. We previously observed that a Dox concentration as low as 10 μM can inhibit the growth of C. innocuum and Lactobacillus. (See FIGS. 7 and 8, respectively).

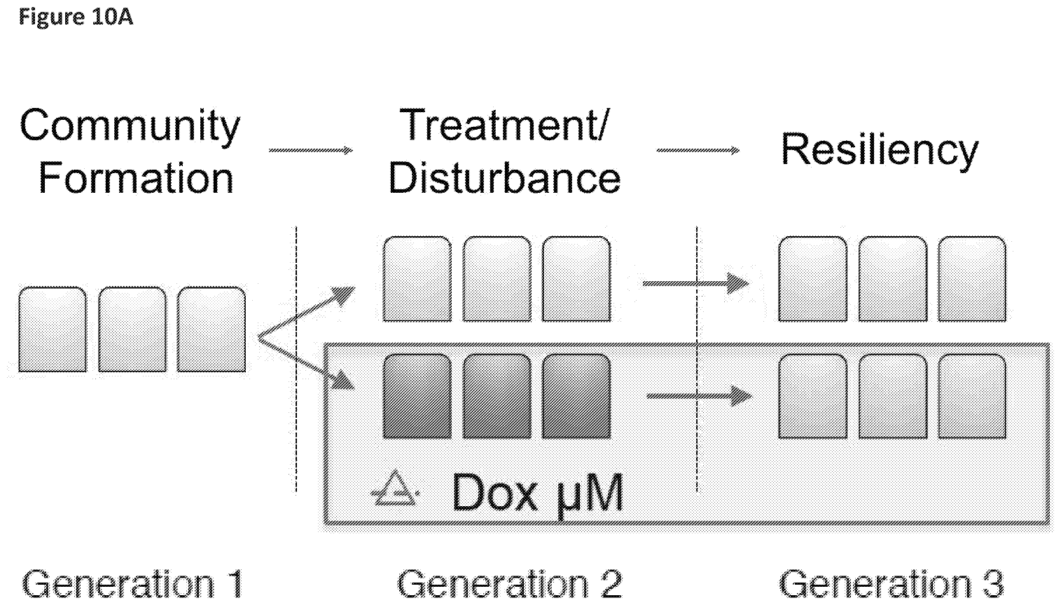

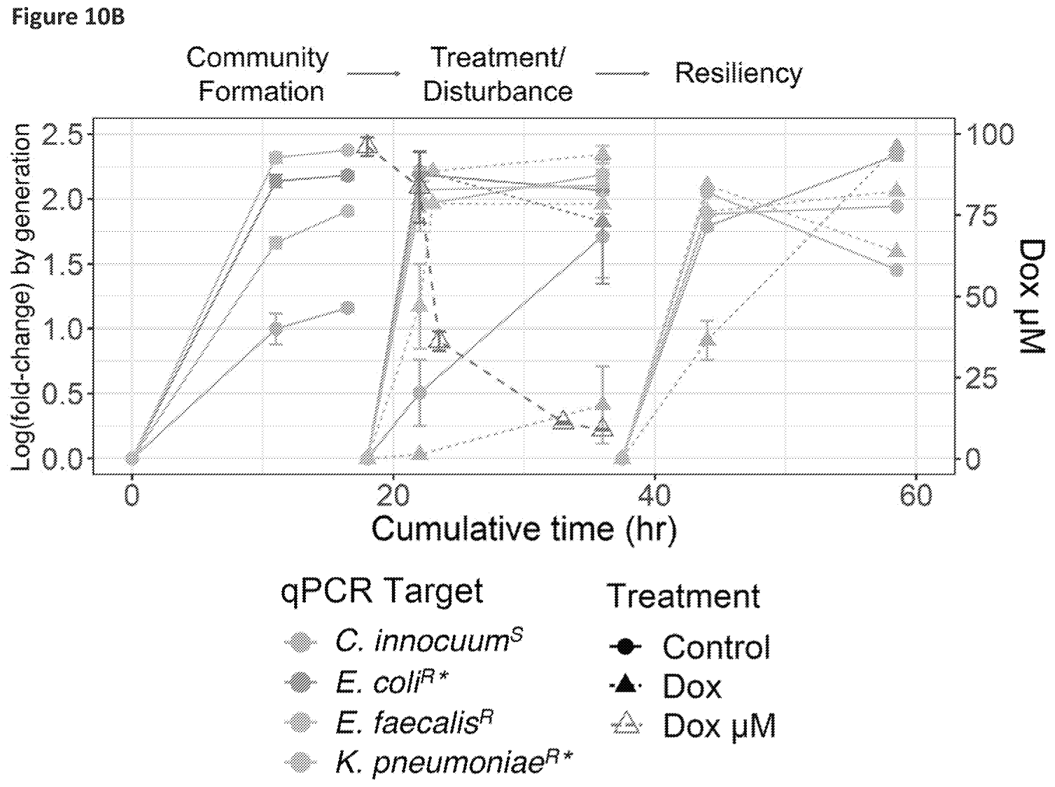

We next performed a mixed-batch culture experiment. (See FIGS. 10A and 10B). We prepared a synthetic community by combining cultures of C. innocuum, E. coli, E. faecalis, and K. pneumoniae. We cultured the synthetic community and allowed community formation in a first generation. Subsequently, we added Dox at a concentration of 100 μM and monitored the growth of the members of the synthetic community after treatment/disturbance in a second generation. We then cultured the synthetic community though resiliency in a third generation.

In our synthetic community in mixed-batch culture, Dox-sensitive C. innocuum grew only following doxorubicin transformation, and the population recovered over time. (See FIGS. 10B, middle panel).

Mechanisms underlying bacterial co-occurrence associations and doxorubicin therapeutic interactions have translational implications. Our future work will focus on key enzymes and probiotic strains with potential use for remediating anthracycline chemotherapeutics to manage mucositis and promote intestinal microbiome diversity during cancer treatment.

### Example 3—Bacterial-Mediated Transformation of Doxorubicin Promotes Growth of Drug-Sensitive Members Within Gut-Associated Bacterial Communities

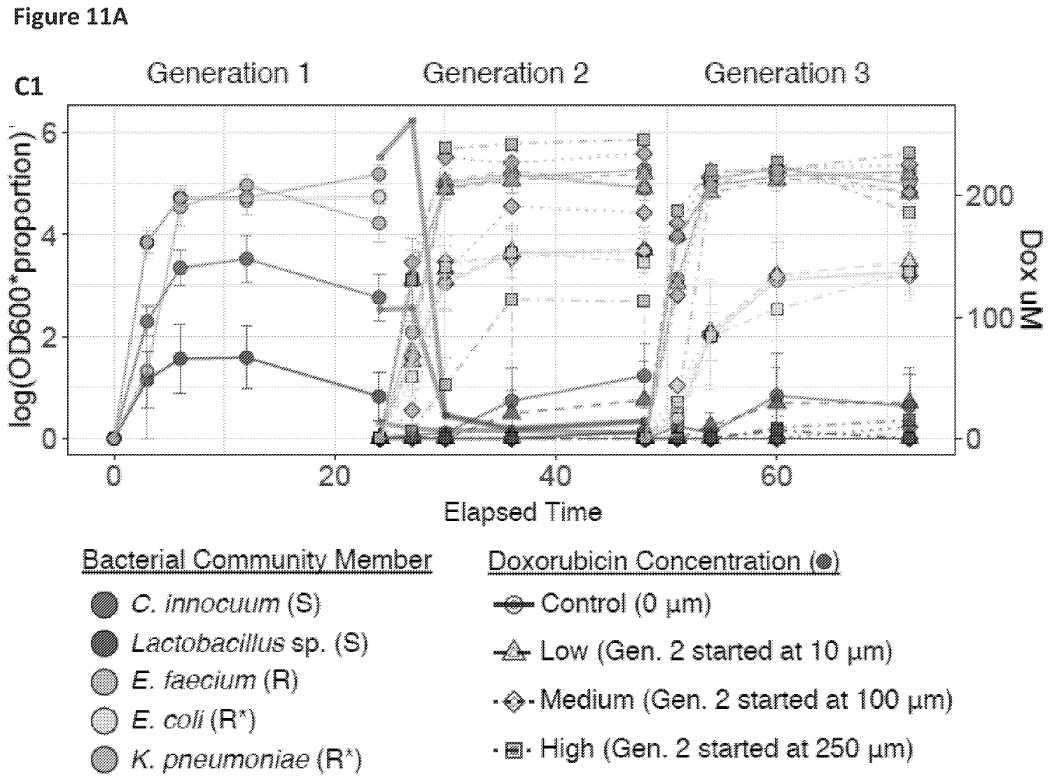

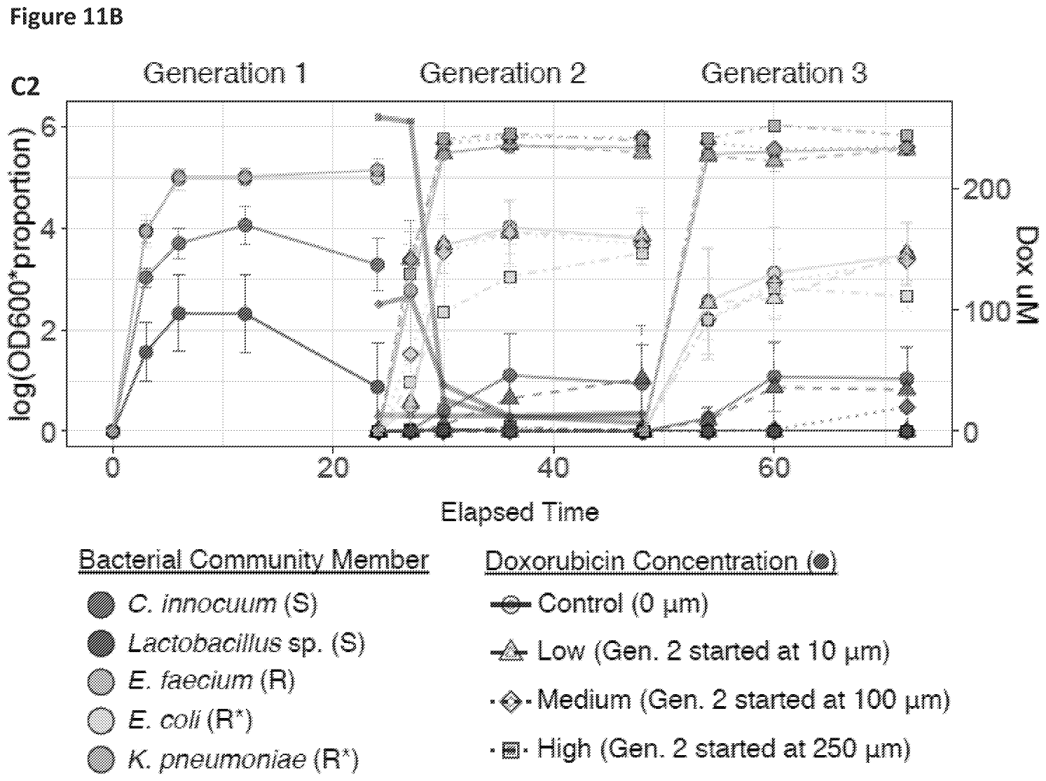

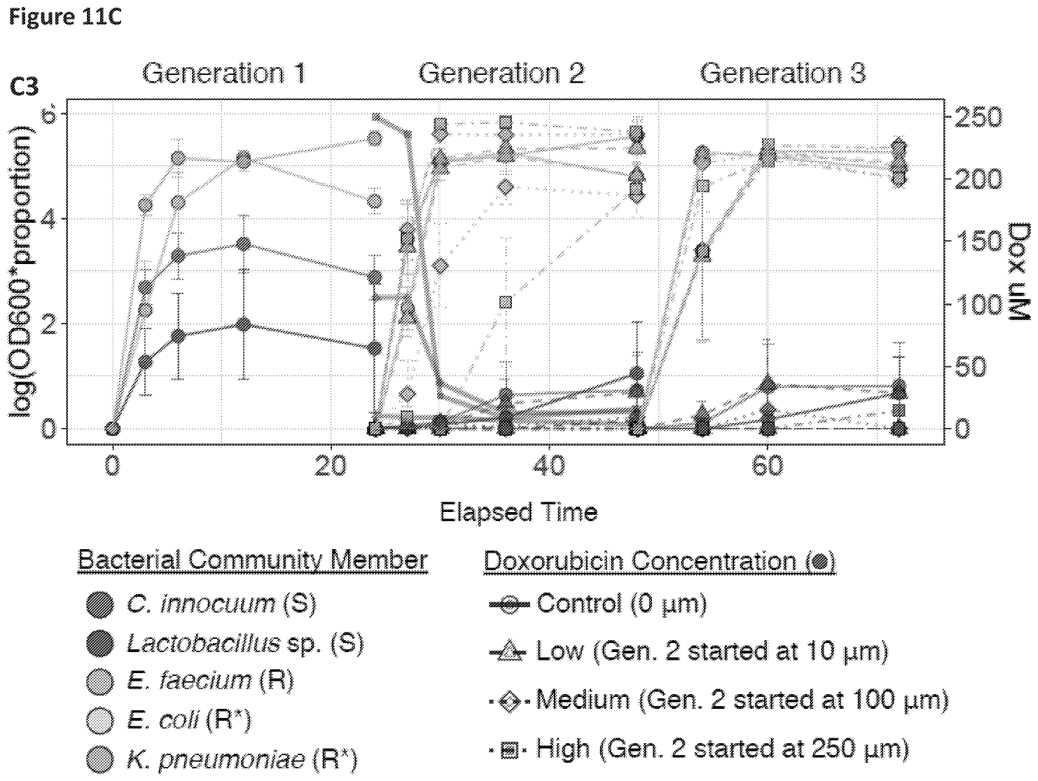

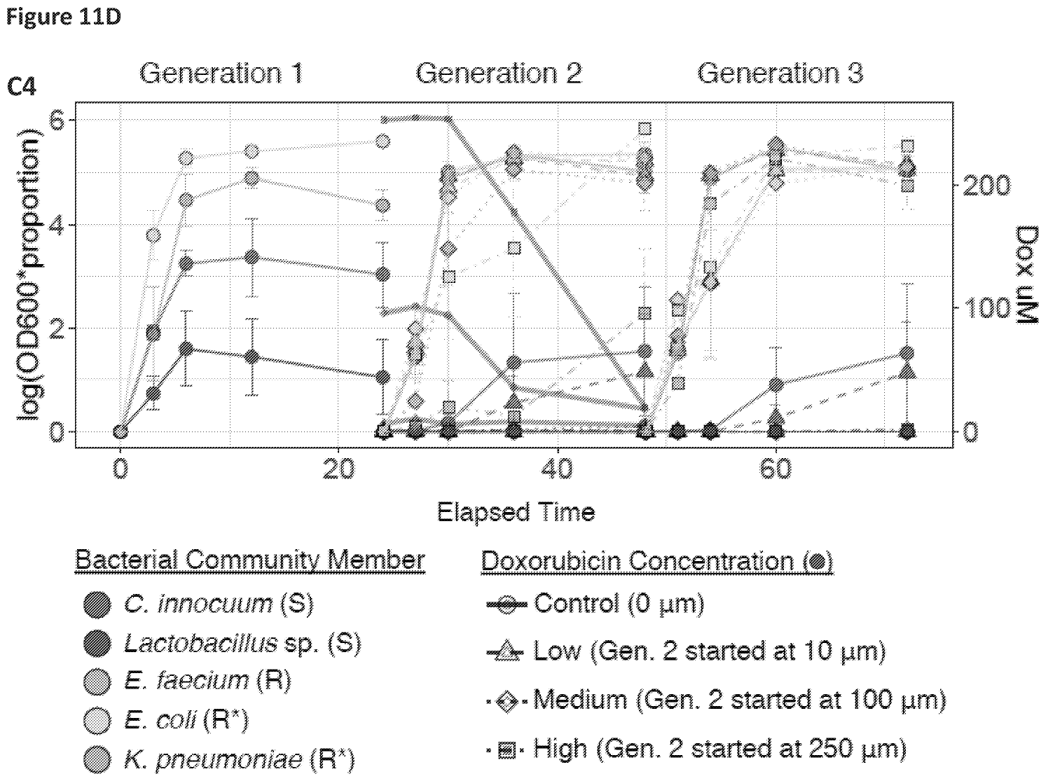

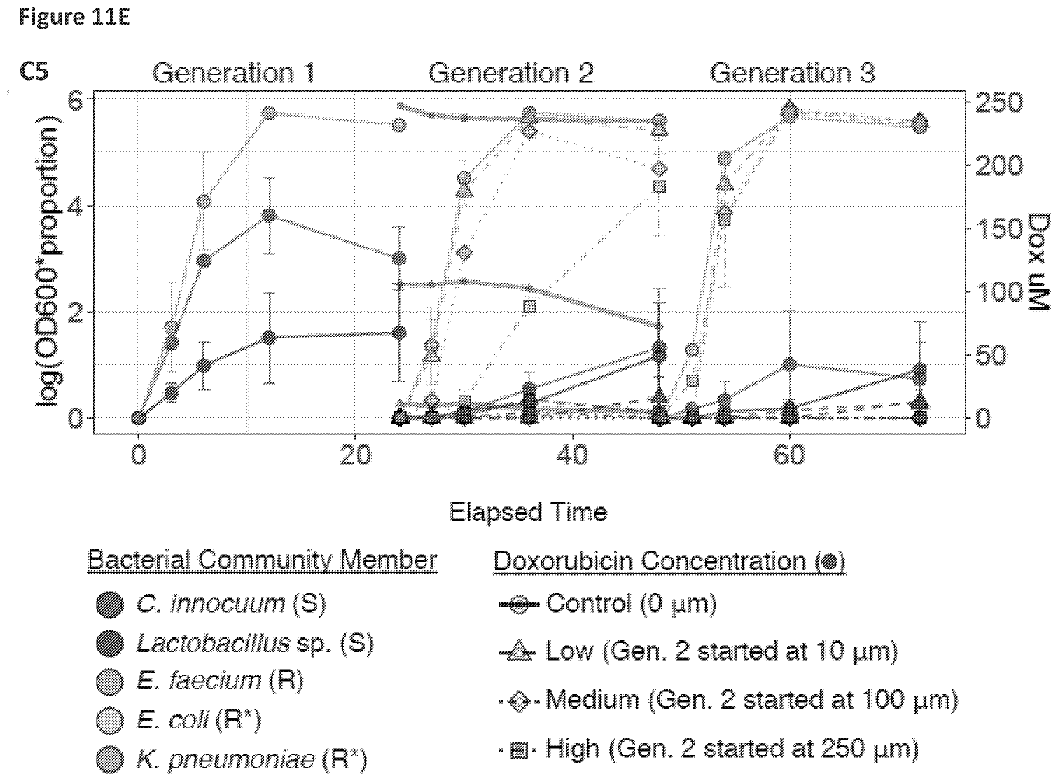

We previously demonstrated that bacterial-mediated transformation of doxorubicin (i.e., an anthracycline chemotherapeutic) promotes growth of drug-sensitive members within gut-associated bacterial communities. The impacts of doxorubicin concentration and microbial community membership (and associated function) on the reduction of bioactive drug and associated microbial community resiliency were investigated in vitro in continuous anaerobic batch culture. Optical density measurements coupled with 16S rRNA gene amplicon sequencing analysis were used to estimate relative bacterial growth and a spectrophotometric assay was used to measure doxorubicin concentration. FIGS. 11A, 11B, 11C, 11D, and 11E shows that bacterial communities containing E. coli or K. pneumoniae carried out rapid transformation of doxorubicin. As illustrated in FIGS. 11A, 11B, 11C, 11D, and 11E we prepared five (5) mixed-microbial communities referred to as C1, C2, C3, C4, and C5, which were composed of members pre-determined to be highly sensitive to doxorubicin (S), resistant via efflux (R), or resistant via efflux and drug-transformation (R*) selected from C. innocuum^{S}, Lactobacillus^{S}sp., E. faecalis^{R}, E. coli^{R*}, and K. pneumoniae^{R*}. C1 began with even content of model strains of C. innocuum^{S}, Lactobacillus^{S} sp., E. faecalis^{R}, E. coli^{R*}, and K. pneumoniae^{R*}. C2 included less E. faecalis^{R}. C3 included less E. coli^{R*}. C4 included less K. pneumoniae^{R*}, and C5 included less E. coli^{R*} and K. pneumoniae^{R*}. The bacterial communities were grown in continuous batch culture and exposed to different concentrations Dox in generation 2.

C1, C2, and C3, may have been enhanced in communities with greater bacterial diversity. (See FIGS. 11A, 11B, and 11C, and compare C1 to C2 and C3, at least for the medium concentration). Bacterial communities containing E. coli^{R*} and lacking K. pneumoniae^{R*} enzymatically transformed Dox at a slower rate. (See FIG. 11D, C4). Bacterial communities without either K. pneumoniae^{R*} or E. coli^{R*} were not able to enzymatically transform Dox. (See FIG. 11E, C5). There were trends suggesting that Dox-transformation rate was associated with resiliency of Dox-sensitive C. innocuum^{S}. Although C. innocuum^{S} growth was not detected in all cultures exposed to medium and high concentrations of doxorubicin (i.e., generation 2 medium and high), populations began to rebound following rapid drug-transformation. (See FIGS. 11A, 11B, and 11C, C1, C2, and C3, generation 3). Such protective effect was not observed in bacterial communities that did not transform the drug (see FIG. 11E, C5) or even in those that had only slowly transformed the drug (see FIG. 11D, C4). Thus, microbial community membership (and associated function) impacted the rate of doxorubicin transformation, which, in turn, may confer protective effects on drug-sensitive members in gut-microbial communities.

We are working on harnessing the observed efficient K. pneumoniae-mediated transformation of doxorubicin for translational purposes in modulating reduction in toxicity of therapeutic that can accumulate in the gastrointestinal tract of patients. Since K. pneumoniae presents inherent risks as an opportunistic pathogen, its use as an active probiotic is suboptimal. Our goal is to apply a variety of approaches to optimize development of safe and effective probiotic interventions for mitigating adverse impacts of anthracycline chemotherapeutics.

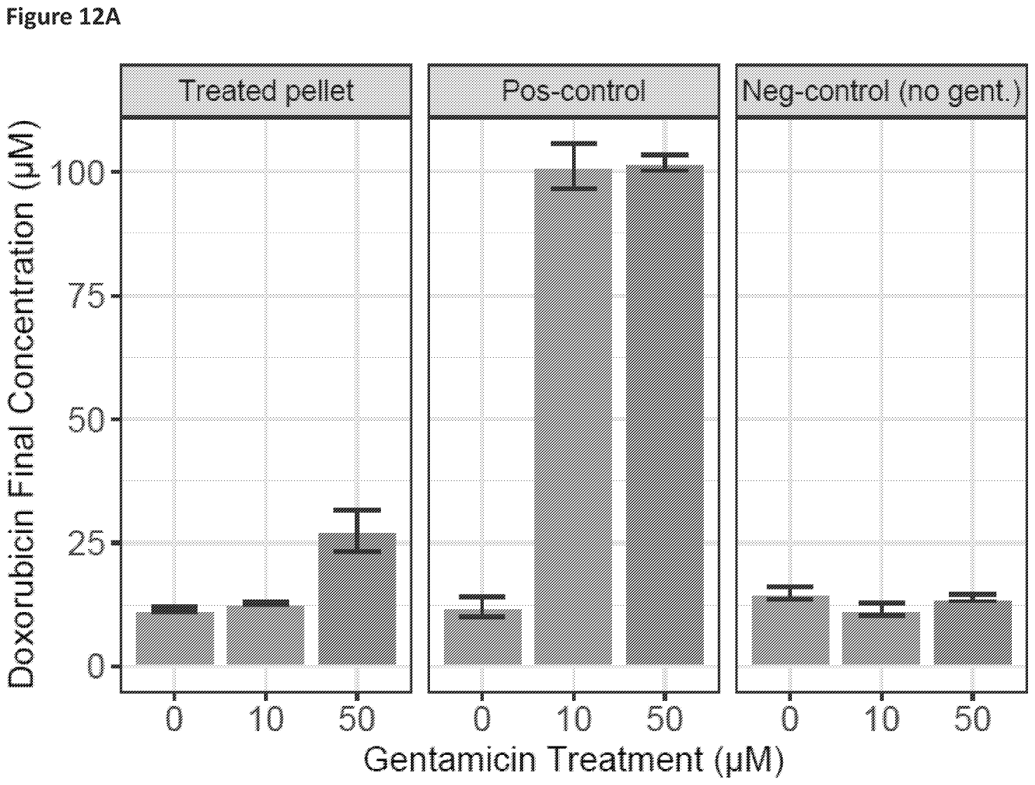

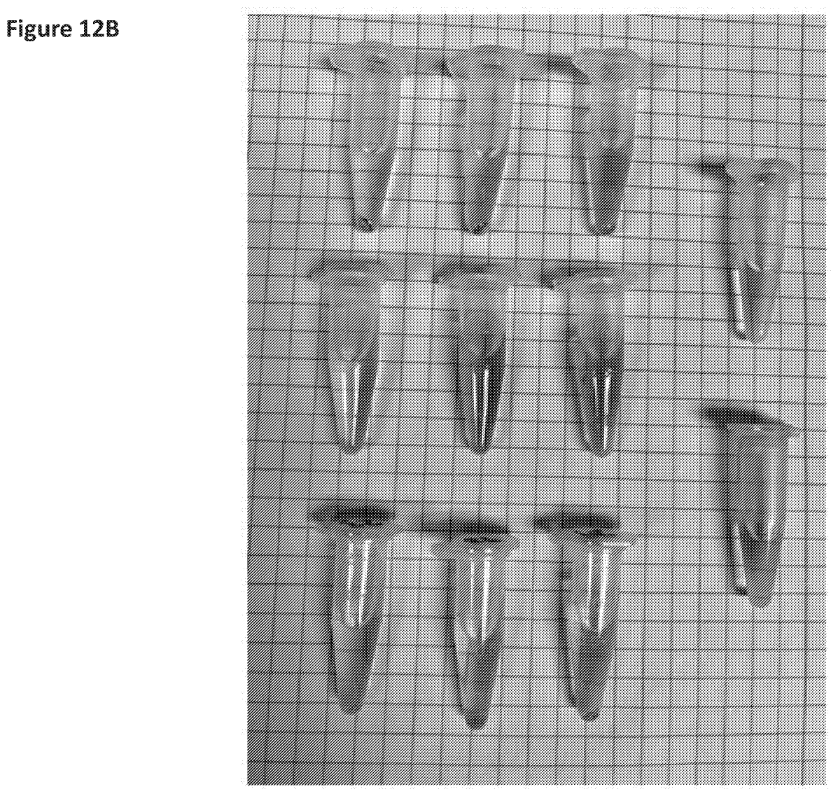

In a modified resting cell assay, we have shown that the biotransformation function can be harnessed without dependency on cell division. (See FIGS. 12A and 12B). Thus, it may be possible to “fix” K. pneumoniae in a form that enzymatically transforms Dox without presenting risk for in vivo colonization/growth. Future efforts will be made to: (1) screen putatively beneficial candidate strains for ability and efficacy in conferring therapeutic transformation (i.e., find alternative options to K. pneumoniae with less risk involved), (2) use genetic engineering techniques to clone the moa genes involved in drug-biotransformation into a harmless alternative strain or to introduce a toxin-antitoxin system to the K. pneumoniae strain to limit its colonization ability, and/or (3) use chemical “fixants” (e.g., glutaraldehyde) to treat the K. pneumoniae strain to remove the ability to replicate while retaining functional activity for enzymatically converting Dox to a less-toxic metabolite.

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references are made herein. The cited references are incorporated by reference herein in their entireties. In the event that there is an inconsistency between a definition of a term in the specification as compared to a definition of the term in a cited reference, the term should be interpreted based on the definition in the specification.

1. A method for treating a subject undergoing treatment with an anthracycline chemotherapeutic or a subject preparing to undergo treatment with an anthracycline chemotherapeutic, the method comprising administering to the subject a detoxifying therapeutic agent that detoxifies the anthracycline chemotherapeutic in the gut of the subject.

2. The method of claim 1, wherein the anthracycline chemotherapeutic is selected from the group consisting of doxorubicin, daunorubicin, epirubicin, and idarubicin.

3. The method of claim 1, wherein the anthracycline chemotherapeutic is doxorubicin.

4. The method of claim 1, wherein the subject is undergoing treatment for cancer or is preparing to undergo treatment for cancer by administration of the anthracycline chemotherapeutic.

5. The method of claim 1, further comprising administering the anthracycline chemotherapeutic to the subject.

6. The method of claim 5, wherein the detoxifying therapeutic agent is administered to the subject before the anthracycline chemotherapeutic is administered to the subject.

7. The method of claim 5, wherein the detoxifying therapeutic agent is administered to the subject concurrently as the anthracycline chemotherapeutic is administered to the subject.

8. The method of claim 5, wherein the detoxifying therapeutic agent is administered to the subject after the anthracycline chemotherapeutic is administered to the subject.

9. The method of claim 5, wherein the detoxifying therapeutic agent is administered to the subject before, concurrently with, and after the anthracycline chemotherapeutic is administered to the subject.

10. The method of claim 1 wherein the detoxifying therapeutic agent comprises one or more enzymes that catalyze metabolism of the anthracycline chemotherapeutic.

11. The method of claim 10, wherein the enzyme is a molybdopterin-dependent enzyme.

12. The method of claim 1 wherein the detoxifying therapeutic agent comprises one or more probiotic organisms that express one or more enzymes that catalyze metabolism of the anthracycline chemotherapeutic.

13. The method of claim 12, wherein the one or more probiotic organisms express one or more molybdopterin-dependent enzymes.

14. The method of claim 1, wherein the detoxifying therapeutic agent is administered orally.

15. The method of claim 1, wherein the detoxifying therapeutic agent is administered gastrointestinally.

16. The method of claim 1, wherein the method treats and/or prevents mucositis and/or promotes microbiota diversity in the subject.

17. The method of claim 1, wherein the detoxifying therapeutic agent is prepared by a method comprising: (a) culturing a bacterial sample obtained from a gastrointestinal tract of a subject in the presence of an anthracycline chemotherapeutic to prepare a cultured sample comprising one or more bacteria that are resistant to the anthracycline chemotherapeutic; and (b) formulating the cultured sample for administration to a subject in need thereof as the detoxifying therapeutic composition.

18. The method of claim 17, wherein the anthracycline chemotherapeutic is doxorubicin and wherein the bacterial sample is cultured in the presence of doxorubicin at a concentration of at least about 50 μM.

19. A method for preparing a therapeutic composition, the method comprising: (a) culturing a bacterial sample obtained from a gastrointestinal tract of a subject in the presence of an anthracycline chemotherapeutic to prepare a cultured sample comprising one or more bacteria that are resistant to the anthracycline chemotherapeutic; and (b) formulating the cultured sample for administration to a subject in need thereof as the therapeutic composition, the method optionally further comprising administering the therapeutic composition to the subject in need thereof.

20. A kit comprising: (i) a detoxifying therapeutic agent that detoxifies the anthracycline chemotherapeutic in the gut of the subject; and (ii) an anthracycline chemotherapeutic.
